(19) United States
(12) Patent Application Publication
(10) Pub. No.: US 2026/0131131 A1
(43) Pub. Date: May 14, 2026

(54) DEVICES AND METHODS FOR SUPPORTING CARDIAC FUNCTION

(71) Applicant: Corisma Cardiovascular, Hamden, CT (US)

(72) Inventors: Mark Kelley, North St. Paul, MN (US); Jim Kelley, Hamden, CT (US)

(73) Assignee: Corisma Cardiovascular, Hamden, CT (US)

(21) Appl. No.: 19/444,401

(22) Filed: Jan. 9, 2026

Related U.S. Application Data
(63) Continuation of application No. PCT/US2024/037471, filed on Jul. 10, 2024.
(60) Provisional application No. 63/513,145, filed on Jul. 12, 2023.

(51) Int. Cl. A61M 60/414; A61M 60/13; A61M 60/174; A61M 60/216; A61M 60/419; A61M 60/508; A61M 60/824; A61M 60/829; A61M 60/861; A61M 60/865

(52) U.S. Cl. CPC A61M 60/414; A61M 60/13; A61M 60/174; A61M 60/216; A61M 60/419; A61M 60/508; A61M 60/824; A61M 60/829; A61M 60/861; A61M 60/865; A61M 2205/3331; A61M 2210/125

(57) ABSTRACT

Systems, devices and methods are provided for supporting cardiac function. A device comprises a catheter having a shaft with a distal end portion configured for advancing through an aortic valve to a left ventricle of a patient and an impeller coupled to the distal end portion of the catheter. One or more inlets are positioned distal of the impeller and coupled to an internal lumen within the catheter to draw blood from the left ventricle through the inlets and into the internal lumen. One or more outlets positioned proximal of the impeller and coupled to the internal lumen to pump the blood through the outlets and into the aorta. The device further includes an extracorporeal motor coupled to a proximal end portion of the catheter and a drive member extending through the catheter shaft and coupling the impeller with the motor to rotate the impeller. The motor may comprise a non-contact motor that generates a magnetic field to rotate the drive member.

### CROSS-REFERENCE TO RELATED APPLICATIONS

This application is a continuation of International Application No. PCT/US 2024/037471 filed Jul. 10, 2024, which claims priority to U.S. Provisional Patent Application Ser. 63/513,145 , filed Jul. 12, 2023, and titled “DEVICES AND METHODS FOR SUPPORTING CARDIAC FUNCTION”, the complete disclosures of which are incorporated herein by reference for all purposes.

### BACKGROUND

A ventricular assist device (VAD) is a medical device that partially or completely replaces the function of a damaged or failing heart. VADs typically assist and unload cardiac chambers of the heart and do not completely take over cardiac function or require removal of the patient's heart. They are used in, for example, patients suffering from a failing heart and in patients at risk for deterioration of cardiac function during percutaneous coronary interventions. A particular VAD may be used to assist the patient's right ventricle (RVAD), left ventricle (LVAD) or both ventricles (BiVAD), depending on the needs of the patient. Such assist devices are either designed to be permanently implanted or they are delivered through the patient's arterial system via a catheter for temporary placement within the heart chamber.

Mechanical circulatory support (MCS) systems have gained greater acceptance for the treatment of acute heart failure, such as to stabilize a patient after cardiogenic shock, during treatment of acute myocardial infarction (MI) or decompensated heart failure, or to support a patient during a high risk percutaneous coronary intervention (PCI). In a conventional approach, a blood pump is inserted into the body and connected to the cardiovascular system, for example, to the left ventricle and the ascending aorta to assist the pumping function of the heart. The ventricular assist device typically includes a tube, which traverses the subject's aortic valve, such that a proximal end of the tube is disposed in the subject's aorta and a distal end of the tube is disposed within the subject's left ventricle. The impeller, the axial shaft and the frame are disposed within a distal portion of the tube inside the subject's left ventricle. The tube typically defines one or more blood inlet openings at the distal end of the tube, via which blood flows into the tube from the left ventricle, during operation of the impeller. For some applications, the proximal portion of the tube defines one or more blood outlet openings, via which blood flows from the tube into the ascending aorta to provide mechanical support to the left ventricle.

While these MCS devices have proven effective in treating certain conditions, there is room for improvement. For example, one of the challenges with these devices is that the impeller must rotate at relative high speeds in order to pump a sufficient amount of blood from the ventricle to the aorta to adequately support cardiac function. To accomplish this, conventional MCS devices have driveshafts between the motor and the impeller that rotate at high speeds. At the same time, the motor required to drive the impeller must be small enough to be delivered through the patient's arterial system to a position proximal of the aortic value in order to drive the impeller. Designing motors small enough to reach the target location in the patient and powerful enough to drive the impeller at the optimal speed has been challenging as these miniature motors are expensive and prone to failure and/or overheating. In addition, the vibration, noise and heat from the motor and the driveshaft can cause discomfort to the patient.

Another challenge with existing MCS devices is that the left ventricle is constantly expanding and contracting, which creates powerful forces on the distal portion of the catheter (i.e., the pump) that is placed therein. In addition, the MCS pump itself is rotating at high speeds, which generates forces that can displace the pump from its intended location. These forces can lead to device migration within the heart chamber, which results in ineffective circulatory support and unloading, as well as hemolysis and arrhythmias. In addition, the impeller or suction head of the device may adhere to the internal walls of the ventricle, which can irritate the heart and lead to obstructions of the pumps inlets, reducing the effectiveness of the device.

What is needed, therefore, are improved devices for supporting cardiac function that overcome the challenges and deficiencies with existing devices. It would be particularly desirable to provide intracardiac devices that reduce or eliminate the existing challenges with conventional motors that drive the pump. It would also be desirable to provide improved devices and methods that reduce migration of the pump during operation within the heart chamber.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

The present disclosure provides systems, devices and methods for supporting cardiac function. The systems and methods are particular useful for use as mechanical circulatory support devices (MCS) to provide hemodynamic support to patients to unload the ventricle and decrease myocardial oxygen consumption. The devices may be useful for temporarily assisting the pumping function of the heart during, for example, angioplasty and/or stent placement procedures (e.g., protected PCI) or for patients who present with, for example, cardiogenic shock, ST-elevation myocardial infarction, cardiomyopathy, fulminant myocarditis, severe heart failure and the like.

In one aspect, a system for supporting cardiac function comprises a catheter having a shaft with a distal end portion configured for advancing through an aortic valve in a patient to a left ventricle and an impeller coupled to the distal end portion of the catheter. One or more inlets are positioned distal of the impeller and coupled to an internal lumen within the catheter to draw blood from the left ventricle through the inlets and into the internal lumen. One or more outlets are positioned proximal of the impeller and coupled to the internal lumen to pump the blood through the outlets and into the aorta. The device further includes an extracorporeal motor coupled to a proximal end portion of the catheter and a drive member extending through the catheter shaft and coupling the impeller with the motor to rotate the impeller.

The extracorporeal motor provides a number of advantages. Since the motor is not delivered into the patient with the pump, it may be designed at a relative low-cost, but with sufficient power and speed to effectively and efficiently drive the pump. The motor may also be designed as a disposable or reusable device that eliminates the need for expensive capital equipment to drive the system. In addition, this design eliminates the need for providing a separate cooling line through the catheter to reduce the heat of the motor. Moreover, providing the motor outside of the patient ensures that the vibration, noise and heat from the motor remains external to the patient, thereby reducing or eliminating patient discomfort during operation of the pump.

In embodiments, the drive member comprises a flexible transmission line, such as a torque cable or the like, configured to impart rotational force on the impeller. In embodiments, the system further comprises a housing for the motor. The housing may comprise a handheld housing that includes an outer casing configured for gripping and holding by the health care provider or the patient. The transmission line extends from a distal end of the outer casing through the catheter shaft to the impeller.

In embodiments, the system comprises an extracorporeal controller coupled to the motor. The controller may be housed within the same housing as the motor, or it may be disposed in a separately housing that is either wirelessly or directly coupled to the motor housing. The controller may be configured to operate the motor (i.e., turn the motor ON and OFF) and/or control the speed of the motor.

In embodiments, the controller includes a processor configured to switch the motor from an activated mode, wherein the motor rotates the impeller, to a deactivated mode, wherein the motor is inoperable to drive the impeller. The activated mode is configured for a defined time period or a defined number of surgical procedures such that the motor will stop working after a defined time period of a defined number of procedures. This ensures that the motor will not be used beyond its useful life. In addition, it may allow the device to be used, for example, on patients after having been prescribed a medical treatment by a physician or other caregiver. The physician may prescribe a defined period of time for the patient to use the medical device and receive treatment. When that time period has been completed, the device is switched back to the deactivated mode so that the patient will no longer receive treatment from the device.

In embodiments, the controller and/or the motor comprises a storage medium that stores a first content and a reader configured to read a second content from the storage medium. The processor is configured to switch the motor from the activated mode to the deactivated mode based on the first content corresponding to the second content. In this manner, the motor may be “filled” with an initial time period and/or an initial number of procedures. The motor will automatically become deactivated when it has been used for the time period of the number of procedures.

In some embodiments, the motor can be capable of being “refilled” with an additional number of procedures or an additional amount of active time by switching the device back to the activated mode. This allows the physician or caregiver to control the level of treatment that a patient receives with the system.

In embodiments, the catheter further comprises a flush line having a proximal end configured for coupling to a source of fluid, such as saline, and a distal opening at the distal end portion of the catheter. The distal opening may, for example, be disposed proximal of the impeller within the pump to flush the pump, thereby removing heat generated by the impeller and minimizing the formation and/or growth of blood clots within the pump.

In embodiments, the system further comprises a fluid purge line having a proximal end configured for coupling to a source of fluid, and a distal opening within the motor for generating positive fluid pressure within the motor. This positive fluid pressure removes heat generated by the motor and prevents ingress of blood into the motor.

In embodiments, the catheter further comprises a hydrodynamic bearing proximal to the impeller and configured to at least partially resist radial forces generated by the impeller during pump operation to provide radial stability to the pump. In certain embodiments, the hydrodynamic bearing is also designed to provide axial stability to the impeller by at least partially resisting axial forces applied by the impeller during operation of the pump. This hydrodynamic bearing reduces or completely eliminates the requirement for magnetic bearings to maintain the axial and/or radial position of the impeller within the catheter. This, in turn, reduces or eliminates the need for additional magnetic coils and associated electronics for such magnetic bearings, thereby providing a more compact axial pump with a reduced footprint within the heart chamber.

In one such embodiment, the hydrodynamic bearing comprises a substantially cylindrical end portion of the catheter disposed within the pump housing proximal to the impeller. This catheter end portion is sized to define an annular gap between the pump housing and the bearing. Fluid may be delivered through the flush line into the annular gap to create a fluid bearing at the proximal end of the pump.

In embodiments, the distal end of the catheter is preformed into a curve, or is configured to deform into a curve. The curve may, for example, substantially correspond to the curvature of the aortic arch of the patient. This facilitates delivery of the pump through the aortic arch and the aortic value into the left ventricle.

In embodiments, the distal end portion of the pump comprises an atraumatic distal tip positioned distally of the inlets. The distal tip minimizes contact between the pump inlets and the internal walls of the left ventricle. In one such embodiment, the distal tip may be configured to move between a substantially linear configuration to an expanded configuration. This allows the tip to be easily inserted through the aortic valve in the linear configuration and then displace into the expanded configuration within the left ventricle to space the inlets of the pump away from the heart walls.

In other embodiments, the housing comprises a wearable device. The housing may include an attachment element, such as a clip, that allows the patient to attach the housing to a wearable garment, such as a belt, pants, skirt, shorts, or the like. Of course, the attachment element may comprise any suitable releasable coupling element, such as fasteners, snaps, interference fit structures, Velcro and the like. Alternatively, the housing may be configured for attachment to a variety of different wearable garments, such as hats, socks, robes, jackets, pants, shirts, vests, shorts, bibs, coveralls, boots, scarves, earmuffs, beanies, underwear, wetsuits and the like, and/or to other non-wearable items, such as blankets, sheets, towels, bandages, seats, mattresses, sleeping bags, and the like.

In another aspect, a system for supporting cardiac function, the system comprises a guide catheter having a shaft with a distal end portion configured for advancing through a blood vessel to an aortic arch within the patient and a pump catheter removably coupled to the distal end portion of the guide catheter. The guide catheter provides controlled positioning and stability for the pump catheter.

In embodiments, the pump catheter comprises an impeller, one or more inlets positioned distal of the impeller and coupled to an internal lumen within the catheter to draw blood from the left ventricle through the inlets and into the internal lumen and one or more outlets positioned proximal of the impeller and coupled to the internal lumen to draw the blood through the outlets and into the aorta. The guide catheter comprises a coupling device that couples the pump catheter to the guide catheter proximal of the outlets. In certain embodiments, the guide catheter is secured to the pump catheter and thus remains within the patient during operation of the pump. In other embodiments, the guide catheter is removably coupled to the pump catheter and can be removed from the patient prior to pump operation.

In one such embodiment, the pump catheter comprises an outer sheath designed for attachment to the distal end portion of the guide catheter. The outer sheath may comprise a proximal hub and an elongate member extending distally from the hub. The pump is preferably disposed within the elongate member. The elongate member has a diameter smaller than the hub and is configured to advanced past the aortic value and into the left ventricle. The hub comprises a tapered distal surface extending radially inward toward the elongate member and has an outer diameter sized to inhibit distal movement of the hub beyond the aortic valve to provide positioning stability for the pump within the left ventricle.

In another aspect, a system for supporting cardiac function comprises a catheter having a shaft with a distal end portion configured for advancing through an aortic valve in a patient to a left ventricle. An impeller is coupled to the distal end portion of the catheter and configured to draw blood from the left ventricle and into the aorta of the patient. The system further includes a positioning device on the catheter proximal of the distal end portion and configured to secure the impeller at a target location within the left ventricle.

In one embodiment, the positioning device comprises a sheath having an outer diameter sized to secure the sheath in position within an artery proximal to an aortic valve. The sheath may be biased into a curved configuration that substantially matches a curve of an aortic arch of the patient. In certain embodiments, the impeller is coupled to the sheath and configured to advance through at least a portion of the sheath to a position distal of the sheath. The sheath inhibits movement of the impeller within the left ventricle during operation.

In another embodiment, the positioning device comprises a wire configured to advance through an internal lumen of the catheter and through an opening in the distal end portion of the catheter. The wire may have a distal end portion configured to expand into an enlarged configuration upon advancement through the opening of the catheter. The opening of the catheter may be distal to the aortic valve and the enlarged configuration of the distal end portion is sized to inhibit proximal movement of the coil through the aortic valve. The impeller is coupled to the wire such that the wire inhibits movement of the impeller in the left ventricle during operation.

In another aspect, a system for treating a medical condition comprises a medical device configured for advancement into a target location within a patient and a drive member having a distal end portion coupled to the device and configured to drive the device. The drive member comprises one or more magnets at a proximal end. The system further includes an energy source spaced from the drive member and configured to generate a magnetic field to rotate the magnets and the drive member. The drive member may comprise a transmission line such as a torque cable or similar device extending to the target location to deliver rotational energy from the energy source to the medical device.

In embodiments, the device further includes an extracorporeal housing and the energy source is disposed within the housing. The device may include a coupling device for removably coupling the proximal end of the drive member to the housing. Alternatively, the coupling device may be part of the housing such that the drive member is removably coupled to the housing.

In certain embodiments, the device further includes a magnetic coil within the housing. The magnetic coil is coupled to the energy source, which generates an electric current through the coil to produce a magnetic field that rotates the magnets and the drive member. In embodiments, the proximal end portion of the drive member extends through an interior of the housing and the coil at least partially surrounds the drive member.

In other embodiments, the device further comprises one or more magnets within the housing. The energy source comprises a motor that rotates the magnets within the housing. The magnets in the housing are positioned and configured to rotate the magnets on the drive member.

In embodiments, the drive member is rotatably coupled to the coupling device and the coupling device comprises a mating feature for non-rotatably attaching the coupling device to the housing. This secures the drive member to the housing and inhibits rotation of the housing during operation.

In embodiments, the device comprises pump with an impeller and the drive member is coupled to the impeller to rotate the impeller at the target site within the patient. The device may further include a catheter configured for percutaneous advancement into a left ventricle of a patient for supporting cardiac function.

In embodiments, the housing comprises a proximal port configured for attachment to a fluid delivery line and an internal fluid channel. The coupling device comprises an internal passage fluidly coupled to the internal fluid channel of the housing. The drive member comprises a fluid channel coupled to the passage within the coupling device for delivering fluid through the drive member to the pump to, for example, create positive fluid pressure within the pump to remove heat generated by the impeller and minimize the formation and/or growth of blood clots within the pump.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1 illustrates a system for supporting heart function;

FIG. 2 illustrates a pump catheter of the system of FIG. 1;

FIG. 3 is a partially cut-a-way view of an external motor of the pump catheter of FIG. 2;

FIG. 4 illustrates a pump on a distal end portion of the pump catheter;

FIG. 5 illustrates an impeller of the pump of FIG. 2;

FIG. 6 is an enlarged view of the impeller;

FIG. 7 illustrates a bearing for the impeller;

FIG. 8 illustrates a proximal end portion of the impeller coupled to the bearing;

FIG. 9 illustrates a system for advancing the pump into the left ventricle of a patient's heart;

FIG. 10 is an enlarged view of the pump positioned within the left ventricle;

FIG. 11 illustrates a positioning wire for the pump

FIG. 12 illustrates a combined guide catheter and pump catheter coupled together;

FIG. 13 is an alternative embodiment of a combined guide catheter and pump catheter;

FIG. 14 is another alternative embodiment of a system for supporting cardiac function;

FIG. 15 is a partial view of a housing for a magnetic drive assembly and a coupling member for a drive cable;

FIG. 16 is a partial cross-section view of the housing of FIG. 15;

FIG. 17 is a partially transparent view of the coupling member within the housing;

FIG. 18 is a cross-sectional view of the coupling member and the housing; and

FIG. 19 is a cross-sectional view of the coupling member inserted within the housing.

### DETAILED DESCRIPTION OF THE PREFERRED EMBODIMENTS

Particular embodiments of the present disclosure are described hereinbelow with reference to the accompanying drawings; however, it is to be understood that the disclosed embodiments are merely exemplary of the disclosure and that the disclosure may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure. Well-known functions or constructions are not described in detail to avoid obscuring the present disclosure in any unnecessary detail. It should be understood also that the drawings are not drawn to scale and are not intended to represent absolute dimensions or relative size. Instead, the drawings help to illustrate the concepts described herein.

Systems, devices and methods are provided for supporting cardiac function. In the representative embodiments, the devices are intended for use as mechanical circulatory support devices (MCS) to provide hemodynamic support to patients to unload the ventricle and decrease myocardial oxygen consumption. The devices may be useful for temporarily assisting the pumping function of the heart during, for example, in angioplasty and/or stent placement procedures (protected PCI) or for patients who present with, for example, cardiogenic shock, ST-elevation myocardial infarction, cardiomyopathy, fulminant myocarditis, severe heart failure and the like. However, it will be recognized that the devices of the present disclosure may also be used for longer term treatment in patients suffering from congestive heart failure, for destination therapy (DT), bridge to transplant therapy (BTT) and for any patients with heart failure who are no longer responding to optimal medical management and are not candidates for heart transplant surgery. In addition, the intracardiac systems and devices described herein, or certain components of such systems, may be used in other applications, such as artificial hearts, ECMO devices, implantable heart monitors and defibrillators, pacemakers, or other intracardiac devices.

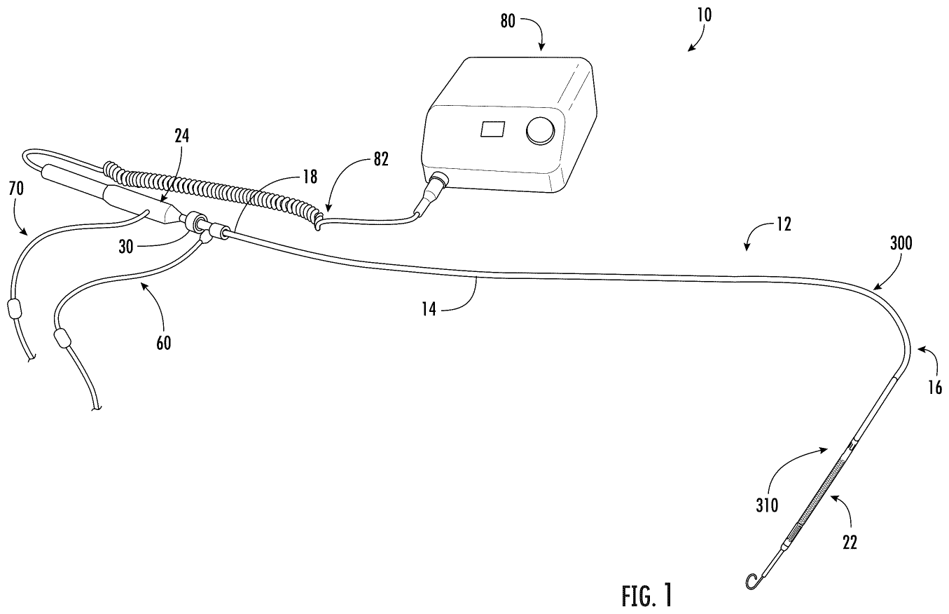

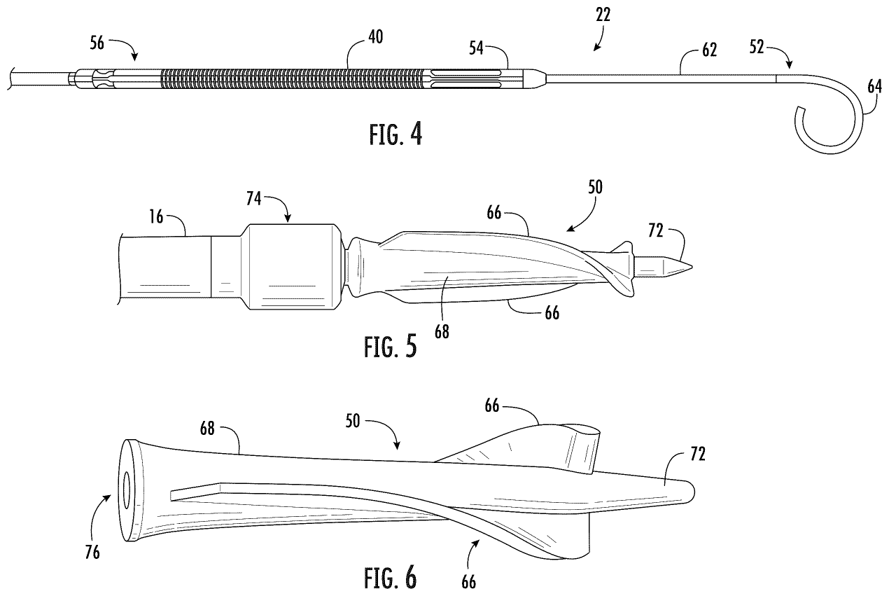

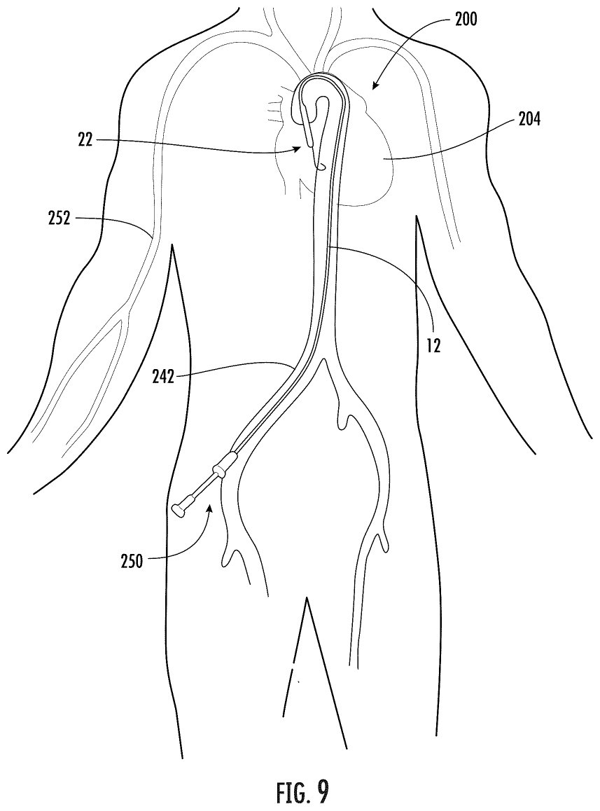

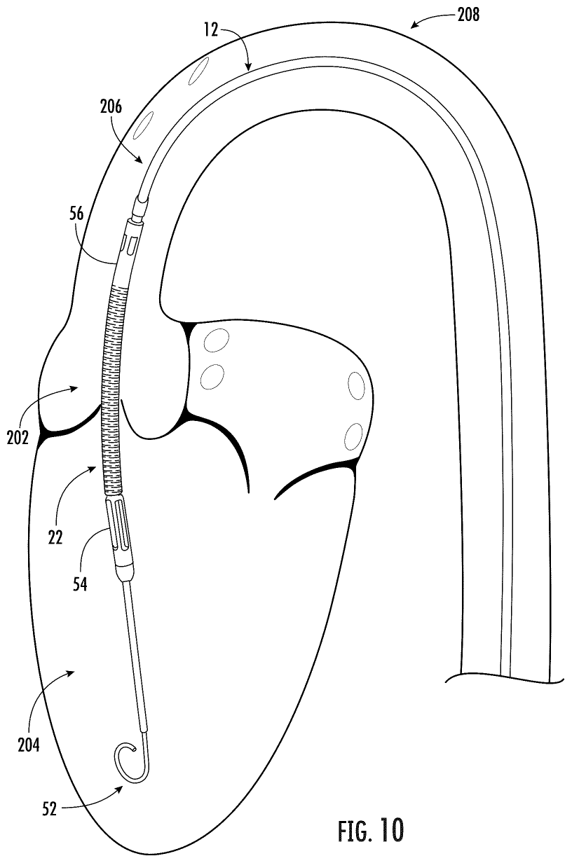

Referring now to FIG. 1, an exemplary ventricular assist system 10 includes a catheter 12 having a flexible shaft 14 with a distal end portion 16 configured for advancement into the patient's arterial system (using an introducer sheath, guide catheter, guidewire, or by itself) across the aortic value 202 and into the left ventricle 204 (see FIGS. 9 and 10 discussed below) of the patient's heart 200. For example, distal end portion 16 of catheter 12 may be introduced into one of the femoral or the brachial arteries. Catheter shaft 14 has a proximal end portion 18 coupled to an extracorporeal housing 24 for a motor 20 (see FIG. 4) for driving a pump 22 on distal end portion 16 of catheter 12. Pump 22 is configured to withdraw blood from the left ventricle 204 of a patient's heart 200 and deliver the blood to the arterial system (i.e., to the aorta 206 as discussed below and shown more clearly in FIGS. 9 and 10). Pump 22 is housed within a casing that may, or may not, have a collapsible stent design.

Motor 20 is positioned within housing 24, which is coupled to proximal end portion 18 of catheter 12 by a coupler device 30. System 10 further includes a transmission line 40 (see FIG. 3) such as a torque cable or similar device extending through an internal lumen (not shown) of catheter 12 from motor 20 to pump 22. Transmission line 40 functions to deliver rotational energy from motor 20 to pump 22 to rotate an impeller 50 (see FIG. 5) within pump 22 to draw blood from left ventricle 204 (discussed in more detail below).

Catheter 12 may have a preformed curve in distal end portion 16. This pre-formed curve may, for example, be designed to substantially match the curve of the aortic arch 208 of the patient to facilitate advancement of the pump 22 through the aortic arch and into the left ventricle. In one embodiment, catheter 12 is formed from an inner liner, a metal (e.g., stainless steel) braid and an outer jacket formed of, for example, polyurethane or polyethylene. The inner liner is designed to provide a smooth or lubricious surface. The braid provides torque and kink resistance and the outer jacket provides support, flexibility and kink resistance.

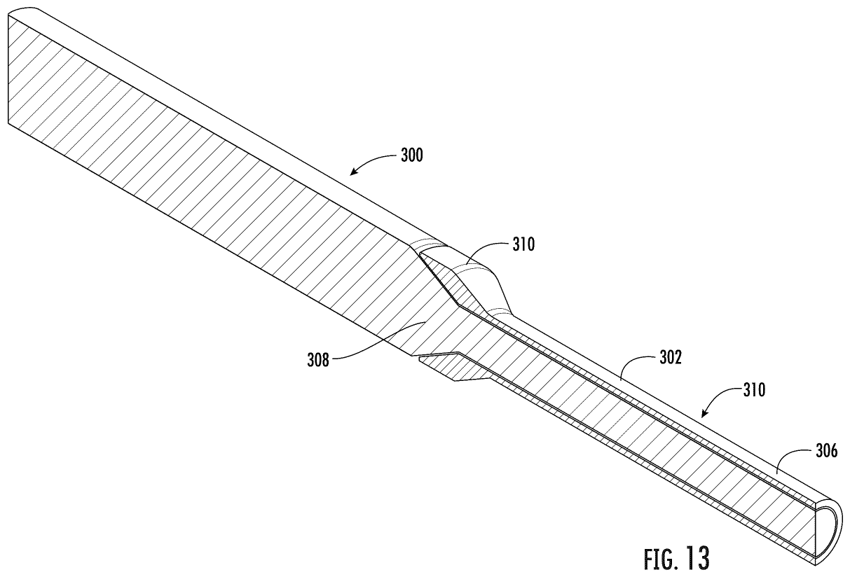

As shown in FIGS. 12 and 13, catheter 12 may comprise a guide catheter 300 configured for percutaneously delivery from an entry point into the femoral or brachial arteries and through those arteries to the aortic arch 208 of the patient. In this embodiment, pump 22 is provided on a pump catheter 310 that is coupled to a distal end portion of guide catheter 300 such that both catheters may be introduced and delivered into the patient at the same time. For example, the catheters may be delivered through the patient's artery system into the aortic arch, and then the pump catheter 310 may be further delivered through the aortic valve and into the left ventricle. In some embodiments, pump catheter 310 is removably coupled to guide catheter 300 such that the guide catheter 300 may be removed from the patient during operation of the pump. In other embodiments, the two catheters remain secured to each other during operation of the pump.

FIG. 12 illustrates one embodiment of catheter 12. As shown, pump catheter 310 includes an outer sheath 302 that surrounds a distal end portion of guide catheter 300. Outer sheath 302 may be secured to guide catheter 300 in any suitable manner. Alternatively, outer sheath 302 may be removably coupled to guide catheter 300 such that pump catheter 310 may remain within the patient while guide catheter 300 is removed. Outer sheath 302 includes an annular surface 304 that tapers inwardly in the distal direction to form an elongate extension 306. Outer sheath 302 of pump catheter 310 is preferably sized such that the distal extension 306 may extend through the aortic valve 202 and into left ventricle 204 (see FIG. 9). At the same time, the proximal portions of outer sheath 302 are preferably sized to remain proximal to the aortic valve 202. This ensures that pump 22 may enter the left ventricle 204, while providing a secure anchor to aortic valve 202 and preventing pump 22 from overextending distally into the left ventricle (i.e., the tapered surfaces 304 of outer sheath 302 provide positional stability to pump 22 in the left ventricle).

In another embodiment (see FIG. 13), guide catheter 300 includes a distal hub 308 coupled to a proximal portion 310 of outer sheath 302 of pump catheter 310. Similar to the previous embodiment, outer sheath 302 of pump catheter 310 is preferably sized such that the distal extension 306 may extend through the aortic valve 202 and into left ventricle 204. At the same time, the proximal portion 310 of outer sheath 302 is preferably sized to remain proximal to the aortic valve 202.

In some cases, it is desirable to infuse a fluid into the pump 22 after the pump has been deployed. For example, the pump 22 can include a system for collecting, purging, or otherwise managing contaminants or debris that can be generated by or come into contact with the working components. To this end, system 10 may further comprise a flush line 60 having a proximal end coupled to a suitable source of fluid (not shown) and a distal end coupled to proximal end 18 of catheter 12 and configured to deliver fluid, such as isotonic saline or the like, through catheter shaft 14 and into pump 22, thereby removing heat generated by impeller 50 and minimizing the formation and/or growth of blood clots within pump 22.

System 10 may further include a motor purge line 70 having a proximal end coupled to a suitable source of fluid and a distal end coupled to an internal portion of motor 20. Purge line 70 functions to generate positive fluid pressure within the motor. This positive fluid pressure removes heat generated by the motor and prevents ingress of blood into the motor.

System 10 may further include a controller housed within an external housing 80 coupled to motor housing 24 by a suitable connector 82. Alternatively the controller may reside in the motor housing, as shown in FIG. 3. Controller may include a processor that communicates with pump 22 to drive motor 20 and control blood flow through the pump. In some embodiments, controller 80 wirelessly communicates with the pump 22. In other embodiments, controller 80 is connected to pump 22 via direct wire connections. In other embodiments, controller 80 is dispersed into several components, one or more of which are integrated into the housing of the heart pump. In some embodiments, significant portions of the control of the motor or pump may reside in controller components that are physically separate from housing 80. In these embodiment, separate components of the controller and motor housings generally communicate with one another wirelessly, although wired or waveguide communication is possible. Thus, the use of wireless technology avoids the inconvenience and distance limitations of interconnecting cables.

The processor may have the ability to monitor the function of the heart pump 22 and/or the cardiac function of the patient. In certain embodiments, controller 80 includes one or more sensing electrodes (not shown) to receive, filter, amplify and analyze an EKG signal. The controller may measure real time function and power consumption of the heart. These measures can then be used to derive many variables of pump function, including speed, flow, suction, pressure head of the pump and an occlusion event. Controller 80 may also have multiple modes, such as a continuous flow mode and/or a pulsatile flow mode, wherein the pump speed is attuned to the systole and diastole periods of the cardiac cycle of the patient. The system may further include an external control unit with a user interface for controlling the specific mode of operation of controller 80, which may include fixed speed (RPM) operation, fixed flow rate operation as well as fixed power operation. A more complete description of one representative controller for use with the system described herein can be found in U.S. Pat. No. 9,919,088, the complete disclosure of which is incorporated herein by reference in its entirely for all purposes.

In some embodiments, external housing 80 may include a user display (not shown) for providing information to the health care provider or the patient related to various parameters of the system, such as the power delivered to the motor 20, the speed of the pump 22 and the like. The user display may also include a user interface that provides input controls for the health care provide or patient to directly modulate certain parameters of the system (discussed below).

The controller may be configured to operate the motor (i.e., turn the motor ON and OFF) and/or control the speed of the motor. In certain embodiments, the controller includes a processor configured to switch the motor from an activated mode, wherein the motor rotates the impeller, to a deactivated mode, wherein the motor is inoperable to drive the impeller. The activated mode is configured for a defined time period and/or a defined number of surgical procedures such that the motor will stop working after a defined time period of a defined number of procedures. This ensures that the motor will not be used beyond its useful life. In addition, it may allow the system to be used, for example, on patients after having been prescribed a specific medical treatment regimen by a physician or other caregiver. For example, the system may be designed to provide support to cardiac function during a specific medical procedure, such as a protected PCI procedure indicated for high-risk complex coronary artery disease intervention for patients without depressed left ventricular (LV) systolic function. A “complex PCI” is typically defined as one having at least one of the following characteristics: (1) 3 or more targeted vessels; (2) 3 or more targeted lesions for treatment; (3) 3 or more stents implanted; (4) a stent length of greater than 60 mm; (5) a target lesion at a bifurcation with at least 2 stents; or (6) a chronic total occlusion of a vessel.

In such a procedure, the motor may be designed for use for a certain number of hours during that procedure, such as about 24 hours, or about 12 hours, or about 8 hours, or about 6 hours, or about 2-4 hours. The physician may prescribe a defined period of time for the patient to use the system and receive treatment. When that time period has been completed, the motor is switched back to the deactivated mode so that the patient will no longer receive treatment from the system.

In embodiments, the controller and/or the motor comprises a storage medium that stores a first content and a reader configured to read a second content from the storage medium. The processor is configured to switch the motor from the activated mode to the deactivated mode based on the first content corresponding to the second content. In this manner, the motor may be “filled” with an initial time period and/or an initial number of procedures. The motor will automatically become deactivated when it has been used for the time period of the number of procedures.

In some embodiments, the motor can be capable of being “refilled” with an additional number of procedures or an additional amount of active time by switching the device back to the activated mode. This allows the physician or caregiver to control the level of treatment that a patient receives with the system.

The reader may be configured to obtain, such as via reading, copying, or others, the content from the storage medium, such as a magnetic card, a radio frequency identification (RFID) card, a chip card, a barcode, a Quick Response (QR) code, or others, such that the processor switches the motor between the first mode and the second mode based on the first content corresponding to the second content, such as logically or others, or vice versa. The first content may also include a second content, such as an activation code, a set of prescription data, a set of dosage/frequency of use data, or others, can be associated with the motor such as uniquely or others, with a specific mode of operation.

Note that the particular patient can be associated with the motor, such as via a primary key of a relational database, as disclosed herein. For example, the primary key can be the PIN or another set of data such that the second content is unique to the particular patient. The second content can be of any of type, whether identical to or different from the first content, such as an alphanumeric, an image, a barcode, a sound, a data structure, a projection, a depression, a hole, or any others. The second content can be formatted in any manner, whether identical to or different from the first content, such as binary, denary, hexadecimal, or others.

The reader or input device can be of any modality or type, such as a camera, a microphone, a sensor, a card reader, a signal receiver, or others. For example, the reader may comprise a reader terminal, that is configured to read the content from the storage medium, such as a card, a display, an interface, a chip, a memory dongle, a paper, or others, whether the storage medium is in or out of a line-of-sight of the reader. For example, when the storage medium is a card, which can include paper, cardboard, plastic, rubber, metal, wood, or others, and the reader is a card reader, then the card can be embedded with at least one of a barcode, a magnetic strip, a computer chip, or another storage medium and the card reader can read the at least one of the barcode, the magnetic strip, the computer chip, or another storage medium. For example, the memory dongle can include a Universal Serial Bus (USB) dongle, a CompactFlash (CF) card, Secure Digital (SD) card, a MultiMediaCard (MMC) card. Therefore, the card can be a dumb card, a smart card, a memory card, a Wiegand card, a proximity card, or others, whether contact or contactless. Correspondingly, the reader can be a smart card reader, a memory card reader, a Wiegand card reader, a magnetic stripe reader, a proximity reader, or others, whether the reader is a non-intelligent reader, a semi-intelligent reader, or an intelligent reader.

In certain embodiments, the input device includes a transceiver, which includes a receiver, that is configured to receive, whether over a wired, wireless, or waveguide connection, the content from the storage medium, such a card, a phone, a tablet, a laptop, a wearable, or others, such via a radio technique, an optical technique, an acoustic technique, or others, whether the storage medium is in or out of a line-of-sight of the transceiver. For example, the radio technique can include a RFID interrogation, a Wi-Fi communication, a Bluetooth communication, or other radio communication formats, which can be encrypted or unencrypted. For example, the optical technique can include a laser beam, an infrared beam, a Li-Fi connection, or others. Note that the transceiver can include a transmitter or a receiver.

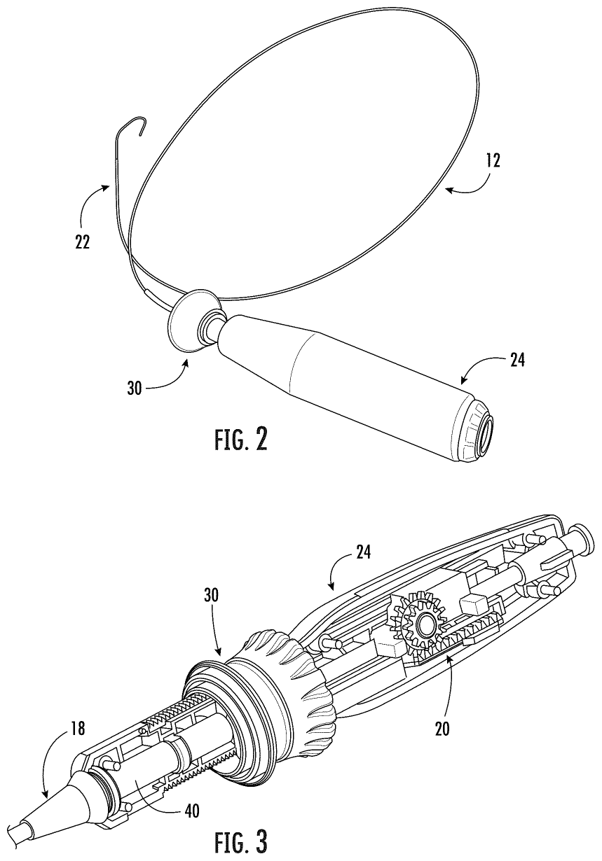

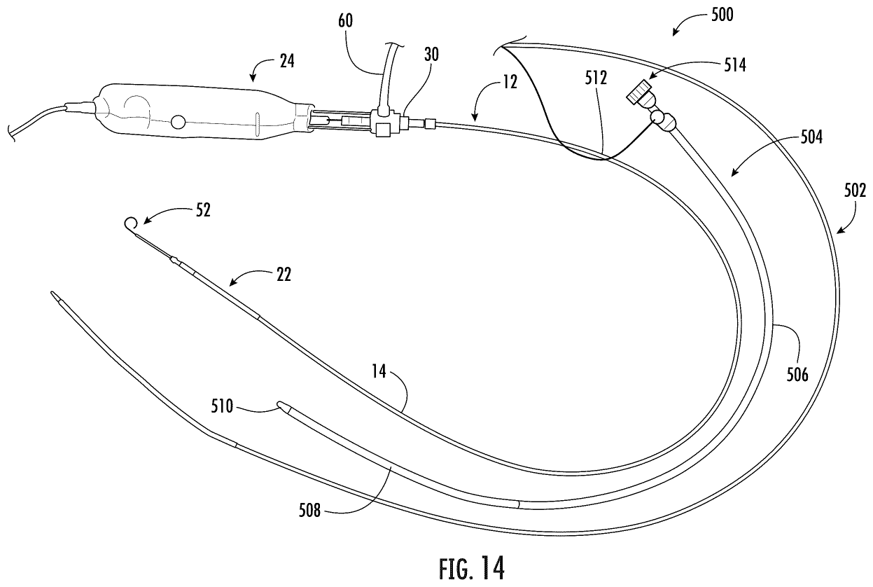

In other embodiments, controller 80 is housed within the motor housing. FIGS. 2 and 14 illustrate an example of this embodiment. As shown, a system 500 is similar to system 10 except that the controller is housed with the motor in housing 24. System 500 comprises a catheter 12 having flexible shaft with a distal end portion 14 configured for advancement into the patient's arterial system (e.g., the femoral or the brachial arteries) across the aortic value 202 and into the left ventricle 204 (see FIGS. 9 and 10 discussed below) of the patient's heart 200. Catheter shaft 12 has a proximal end portion coupled to casing 24 that houses an extracorporeal motor for driving a pump 22 on distal end portion 14 of catheter 12. In this embodiment, the controller is housed within the casing 24 and directly coupled to the motor.

FIG. 2 illustrates one embodiment of a housing 24 for motor 20. Housing 24 may comprise any configuration, but preferably comprises a relatively small, lightweight construction. Housing 24 and motor 20 may be configured as single-use device that is disposable after one use by the patient. In other embodiments, housing 24 and motor 20 are configured for multiple uses by a single patient or multiple patients. In certain embodiments, housing 24 includes a timing control that monitors usage of the motor over, for example, a period of time, or a number of patient uses. The timing control is configured to shut down motor 20 after it has been used for a certain number of times, or uses.

In certain embodiments, the housing 24 may be configured to be handheld by either the user or the caregiver. In other embodiments, housing 24 may comprise a wearable device that can be attached to, or worn by, the patient. In some embodiments, housing 24 also includes an attachment element (not shown) for attaching housing 24 to a patient. The attachment element may comprise any suitable releasable coupling element, such as fasteners, snaps, interference fit structures, Velcro and the like. Housing 24 may be configured for direct attachment to the patient's outer skin surface or for attachment to a variety of different wearable garments, such as pants, belts, chest straps, pendants, sashes, hats, jackets, shirts, vests, shorts, skirts, bibs, coveralls. The housing may also include a waterproof outer shell around to insulate the motor, controller and associated electronic circuits from water or other fluids that may contact the garment.

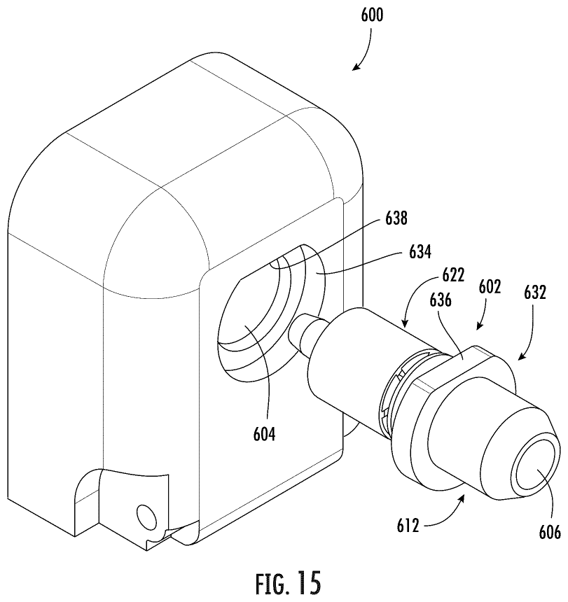

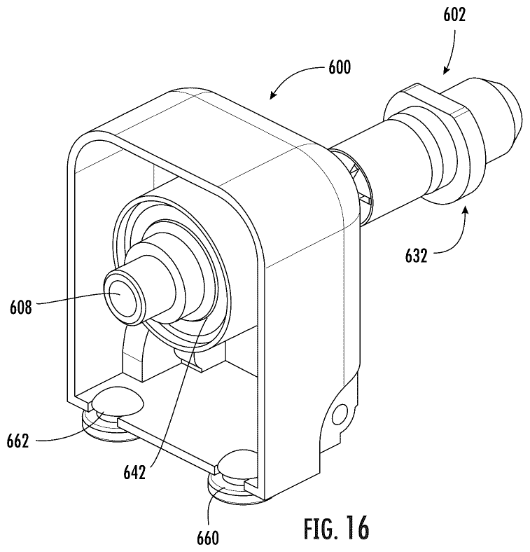

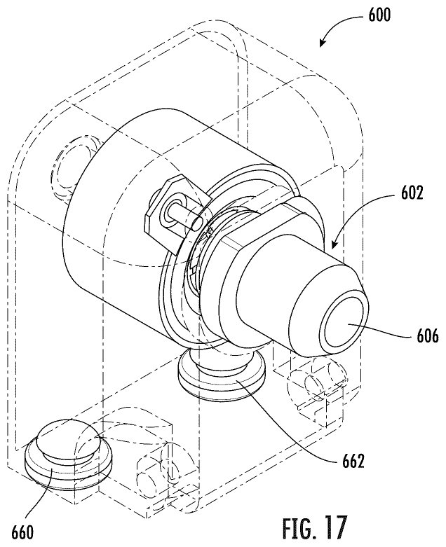

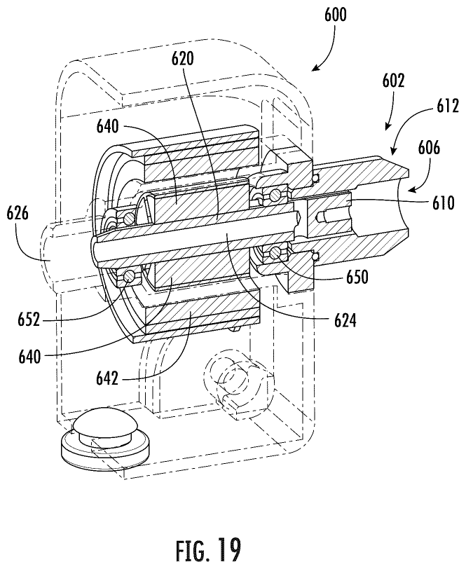

As shown in FIG. 3, motor 20 may comprise any suitable motor. In certain embodiments, motor 20 is designed to function at speeds of up to 35,000 rpms. For example, motor 20 may include a motor stator (not shown) that is preferably integral with outer casing 24 and may include stator windings and a back iron. Motor 20 may further comprise a rotatable element, such as a rotor portion of the motor that is configured to be rotated (i.e., driven) by the motor stator. In one embodiment, the motor stator includes one or more permanent magnets and the rotor includes one or more magnets such that the rotor may be rotated around its longitudinal axis by a suitable magnetic field, as is known in the art. In this embodiment, housing 24 may be formed from a magnetically permeable material selected to minimize power losses due to magnetic hysteresis. Electrical conductors (not shown) passing through housing 24 provide power and control signals to the electric motor. Alternatively, motor 20 may comprise a non-contact motor that is spaced away from transmission line, such as the magnetic motor described below in reference to FIGS. 15-19.

Transmission line 40 may comprise a drive cable that operatively couples motor 20 to pump 22, thereby transmitting motor torque to the pump to drive the impeller. The drive cable preferably rotates at a speed well in excess of 1000 rpm and possibly as high as 100,000 rpm. The motor can be designed to produce high rotation speeds, for example rotation speeds in a region between 10,000 and 40,000 revolutions per minute. The functional element which is connected to the distal end-piece of the drive shaft in a rotationally fixed manner is designed as a pump rotor.

In certain embodiments, drive cable 40 has a substantially rigid portion at its distal end which is connected to pump 22 and a substantially flexible portion extending through catheter shaft 14. The substantially flexible potion extends along a majority of the length of the drive. In one such embodiment, drive cable 40 comprises a multitude of coaxial windings which run spirally around the cavity of the drive shaft, in order to convert torsion and bending stresses into axial tensile and compressive stresses. In other embodiments, the drive cable 40 may include a plurality of discrete segments that are coupled end-to-end, thereby forming a linear arrangement. Each of the segments has a structural configuration that renders it flexible enough to rotate while conforming to a curved path.

In certain embodiments, the segments are configured as coils, which are preferably, but not necessarily, hollow (i.e., have lumens). Compared to a rod configuration, a straight section of coil has a substantially decreased ability to withstand the stresses that result from transmitting torque at high rpm. But a bent section of coil has a substantially greater ability than a rod to withstand the stresses that result from transmitting torque at high rpm around a corner or bend. Thus, flexible segments have a greater capacity for transmitting torque at high rpm along a curved path than rod segment. In various embodiments, the coil segments can be a single coil or multi-layer (i.e., a coil-within-a-coil). Additionally, in some embodiments, the coils are uni-filament and in some other embodiments, they are multi-filament.

Referring now to FIG. 4, pump 22 includes an outer casing 40 for housing an impeller 50 (see FIGS. 5 and 6) that pumps blood from the left ventricle to the aorta. Casing 40 may, or may not, have a collapsible stent design. Outer casing 40, which in some embodiments may comprise a substantially cylindrical body, preferably has a substantially uniform outer diameter to facilitate insertion of the pump into an artery or specific delivery device. Casing 40 further includes one or more inlets 54 distal to impeller 50 for drawing blood from the left ventricle into an internal lumen (not shown) of casing 40, and one or more outlets 56 distal of impeller 50 for pumping the blood from the internal lumen to the aorta. The exact size and shape of inlets 54 are designed to provide sufficient flow from a heart chamber surrounding pump 22 into the aorta.

In one embodiment, casing 40 is formed from a slotted tube formed of any suitable material, such as a metal, plastic or the like. The slotted tube is covered with a sheath such that only the distal and proximal slots are exposed, thereby forming inlets 54 and outlets 56. The outer sheath may be formed of any suitable material, such as a flexible polyethene or pellethane coating or the like. Of course, other configurations are possible. For example, inlets 54 may comprise one or more openings spaced from each other around the circumference of casing 40. Such openings may have any suitable cross-sectional shape, e.g., circular, square, diamond, rectangular, triangular or the like.

Pump 22 further includes a tip element 52 extending distally from casing 40 and configured to separate inlets 54 of pump 22 from the internal walls of left ventricle 202. In the representative embodiment, tip element 52 comprises a substantially linear portion 62 and a distal curved portion 64. In certain embodiments, tip element 52 is configured to separate inlets 54 from a septal wall of left ventricle 202 as curved portion 64 of tip element 52 contacts the apex of left ventricle 202. For example, when tip element 52 is inserted into the left ventricle such that the bulge bulges toward the septal wall, in response to curved portion 64 of tip element 52 being pushed against the apex of left ventricle 202, inlets 54 are pushed away from the septal wall and toward a free wall of left ventricle 202.

In certain embodiments, tip element 52 may be movable between a substantially linear configuration to a partially curved configured (shown in FIG. 4). In some embodiments, the curve is greater than 180 degrees (e.g., 200, 220, 240, 260, 270, 300, 320 degrees or any suitable angle). The tip element 52 may comprise a flexible material, or the tip element 52 may comprise different components having different stiffnesses. For example, linear portion 62 may be more rigid than curved portion 64. The linear portion 62 inhibits distal movement of pump 22, thereby inhibit movement of outlets 56 from moving into the left ventricle of the heart. The flexible curved portion 64 provides an atraumatic tip for contact with the internal walls of the left ventricle.

As shown in FIG. 6, impeller 50 comprises one or more blades 66 extending around a central hub 68 and a distal tip portion 72. Impeller 50 provides an efficient design that may pump at least 2.5 Liters of blood per minute, preferably at least about 4 Liters/minute, at the physiological pressures typically existing within the heart chambers. The blades 66 may take any appropriate shape and be of any appropriate number. Blades 6 preferably define a clearance with the inner surface of casing 40 of about 0.1 mm to about 0.8 mm, preferably about 0.2 mm to about 0.4 mm, more preferably about 0.3 mm. In one embodiment, blades 66 have a substantially helical shape such that the blades 66 spiral around hub 68 from the upstream end to the downstream end. Blades 66 may have the same, or a different, pitch. Each blade 66 may have a pitch that varies from hub 68 to the tip of the blade 66.

As shown in FIG. 6, impeller 50 may include two blades 66 extending from hub 68 and spaced apart from each other. In certain embodiments, the pitch angle of each of the blades 66 changes in the longitudinal direction such that the angle between the blade surface and the blood flow increases in the downstream direction. Thus, the angle between the blade surface at the hub (where the blood first contacts the blade) is smaller and closer to parallel to the blood flow direction to reduce turbulence and minimize damage to the blood cells upon initial contact with blade 66. As the blood flows along the surface of the blade downstream, this angle increases to provide sufficient power to accelerate the blood flow and propel the blood radially relative to the housing.

Impeller 50 may further comprise a stator (not shown) that is configured to redirect the flow of the blood from the radial direction to the longitudinal direction towards outlets 56. The stator may, for example, includes one or more blade-shaped surfaces that have pitch angles that decrease in the downstream direction. Similar to the impeller blades, these surfaces are designed to reduce the impact of the radial blood flow at the upstream end of the surface and then to gradually redirect this blood flow in the longitudinal direction. This design reduces turbulence and minimizes damage to blood cells. The stator may be coupled to impeller 50 with a mechanical bearing or other suitable coupling device. A more complete description of suitable stators for use with the impeller described herein can be found in commonly assigned International Patent Application No. PCT/US0222/035172, the complete disclosure of which is incorporated herein by reference for all purposes.

Of course, the pumps described herein are not limited to the specific impeller configuration described above and shown in the figures. For example, blades 66 can be foldable against hub 68 so as to reduce cross-sectional size of impeller 50 for percutaneous insertion into the body. Once impeller 50 has been located in a desired position, blades 24 can be expanded away from hub 68 to place impeller 50 in operation for pumping blood.

In other embodiments, pump 22 can alternatively employ a fluid actuator that has a shaftless design for the actuation of fluids. The actuator comprises a housing having a plurality of blades. The housing has a hollow, substantially cylindrical shape having a long axis with open ends and an outer and an inner surface. Each of the blades is attached to the inner surface of the housing and extends from opposite ends of housing in a helical pattern. The blades are thereby configured to actuate a fluid by the rotation of the housing along its long axis. The rotation can be achieved by mechanical linkage with a motor, such as by a rim driven connection or an end-driven connection. The rotation can also be achieved by magnetic coupling with external electromagnets or a rotating magnet. The blades may have any suitable cross-section shape, including a substantially parallelogram-like cross-sectional, rectangular, with rounded edges, with sharp edges, and the like. A more complete description of a suitable fluid actuator with a shaftless design can be found in International Patent Application No. PCT/US2019/037047, the complete disclosure of which is incorporated herein by reference in its entirety for all purposes.

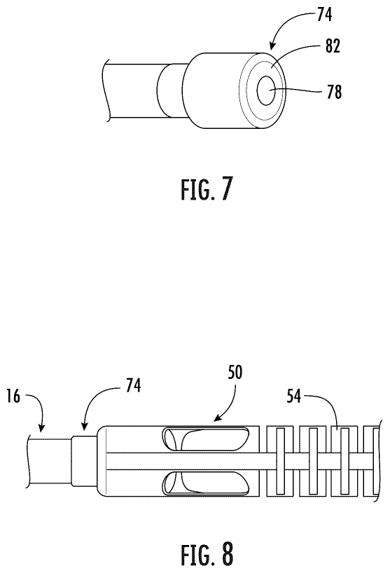

As shown in FIGS. 7 and 8, pump 22 further comprises a mechanical bearing 74 coupled to distal end portion 16 of catheter 12. Bearing 74 is coupled to a proximal end of pump casing 40. Bearing 74 is preferably designed to resist axial and rotational forces applied by and against impeller 50 as impeller 50 pumps blood through pump 22. In one embodiment, bearing 74 also functions as a fluid bearing and is sized to provide an annular gap between an outer surface of bearing 74 and the inner surface of casing 40. Catheter 12 further comprises an internal lumen (not shown) fluidly coupled to the annular gap for delivering a fluid therethrough. The fluid passing through this gap resists axial and/or radial forces applied to pump 22. Alternatively, a pump 22 may include a secondary flow path for blood passing therethrough. The secondary flow path may include the annular gap such that the blood provides the fluid for the fluid bearing.

Catheter 12 may further include additional bearings (not shown) for maintaining the axial and/or radial positions of impeller 50 within casing 40 in the event that the bearing 74 does not sufficiently resist these forces. In one embodiment, for example, a magnetic bearing may comprise a permanent axial housing magnet (not shown) positioning within casing 40 that cooperates with a permanent axial rotor magnet (not shown) positioned in impeller 50. In another embodiment, the magnetic bearing may include an active magnetic bearing that operates alone or in conjunction with a passive magnetic bearing. In this embodiment, the axial magnetic bearing may comprise, for example, a cylindrical passive magnet designed to counteract the axial forces encountered when impeller 50 is up to speed, surrounded by an active magnet, designed to compensate for additional axial loads, such as those present during pre-load or after-load of impeller 50. In yet another embodiment, permanent magnets may be radially distributed around impeller 50. The attractive force of the magnetic coupling provides axial restraint to impeller 50.

Catheter 12 may also include a radial magnetic bearing for stabilizing radial forces against impeller 50 to minimize contact between impeller 50 and casing 40. For example, permanent radial bearing magnets (not shown) may be disposed within casing 40 and designed to cooperate with rotor bearing magnets in impeller 50. The radial bearing magnets allow the impeller 50 to rotate relative to casing 40 without significant radial contact. In addition, they assist the fluid bearing described above to maintain the annular clearance between impeller 50 and casing 40.

In certain embodiments, the system further includes a positioning device on the catheter 12 proximal of distal end portion 16 and configured to secure impeller 50 at a target location within the left ventricle. In one such embodiment, the positioning device comprises an introducer sheath (not shown) having an outer diameter sized to secure the sheath in position within an artery proximal to an aortic valve. The introducer sheath may be biased into a curved configuration that substantially matches a curve of an aortic arch of the patient. In certain embodiments, pump 22 is coupled to the sheath and configured to advance through at least a portion of the sheath to a position distal of the sheath. The sheath inhibits movement of the impeller within the left ventricle during operation.

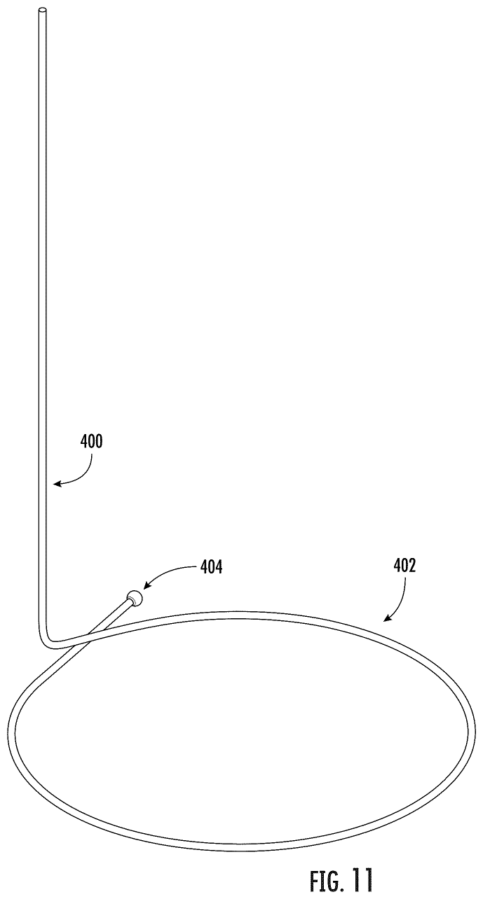

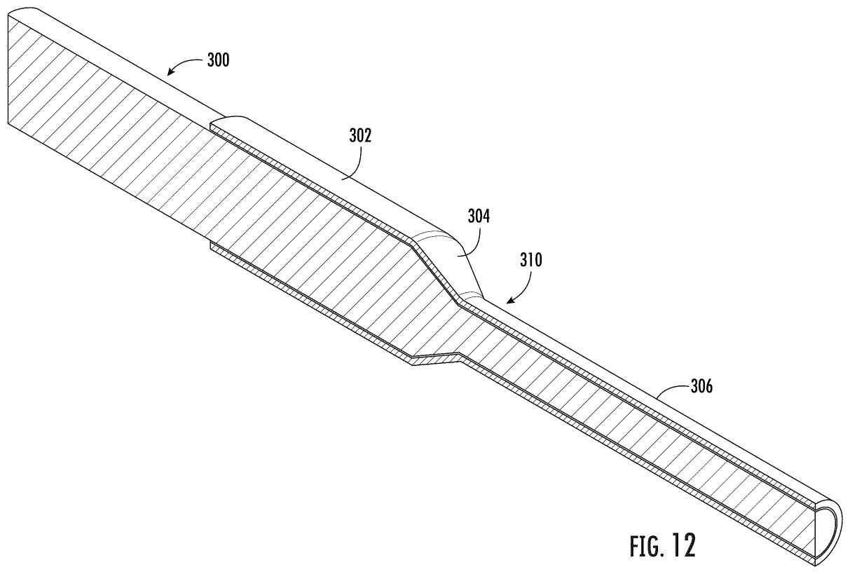

In another embodiment, the positioning device comprises a wire 400 configured to advance through an internal lumen of the catheter 12 and through an opening in the distal end portion of the catheter 12 (see FIG. 11). The wire 400 may have a distal end portion 402 configured to expand from a substantially linear portion into an enlarged configuration upon advancement through the opening of the catheter 12 (as shown in FIG. 11). The opening of the catheter 12 may be distal to the aortic valve and the enlarged configuration of the distal end portion 402 is sized to inhibit proximal movement of the expanded or coiled wire 400 through the aortic valve. The impeller 50 is coupled to the wire 400 such that the wire 400 inhibits movement of the impeller 50 in the left ventricle during operation.

Referring now to FIGS. 9 and 10 systems and methods for delivering pump 22 into a left ventricle 204 of the heart 200 will now be described. In one approach, a guidewire (not shown) is first placed in a conventional manner, e.g., through a needle into a peripheral blood vessel, such as the femoral artery 242 or the brachial artery 252. The guidewire may be advanced along the path between that blood vessel and the heart and into a heart chamber, e.g., into the left ventricle. Thereafter, a distal end opening of a catheter assembly 250 or guidewire guide can be advanced over the proximal end of the guidewire to enable delivery of the catheter assembly. In certain embodiments, catheter assembly 250 comprises both guide catheter 300 and pump catheter 310 (as shown in FIGS. 12 and 13). In other embodiments, the guide catheter 300 is introduced first and the pump catheter 310 is a separate elongate flexible shaft that passes through the guide catheter 300. In either embodiment, the guide catheter 300 may be removed after the pump catheter 310 has been delivered into the left ventricle (or the guide catheter may remain in position if it is attached to the pump catheter 310 as shown in FIGS. 12 and 13).

The pump 22 is delivered distally until the inlets 54 are disposed within the left ventricle 204 and outlets 56 are positioned in the aorta 206 proximal of the aortic valve 202 such that blood can be pumped from the ventricle through the conduit and into the systemic circulatory system. A portion of the pump 22 is centered across the patient's aortic valve 202.

In certain embodiments, an introducer sheath may be used to stabilize the position of the pump 22 within the left ventricle 204. The introducer sheath may be separate from the guide and pump catheters or may be incorporated into the guide catheter and includes a distal sheath sized to be secured into position within the aortic arch 208 proximal of the aortic valve 202. The pump or pump catheter may be advanced distally of the sheath and then locked into position relative to the sheath (or the sheath may be backloaded through the delivery catheter after the pump has been delivered to the left ventricle). The sheath maintains a position of the pump within the left ventricle during use

In other embodiments, wire 400 may be used to stabilize the position of the pump 22 within the left ventricle 204. Wire 400 is configured to advance through an internal lumen of the catheter 12 (or through the pump catheter) and through an opening in the distal end portion of the catheter 12. The wire 400 is expanded into the enlarged configuration shown in FIG. 11 to inhibit proximal movement of the expanded or coiled wire 400 through the aortic valve.

In certain techniques, the pump 10 can be delivered to a treatment site without the use of a guidewire. For example, once access has been provided to the vasculature, the pump 22 can be advanced to the descending aorta by pushing on the proximal end of the device to advance the distal end along the peripheral vessels (e.g., femoral or iliac), to track through a portion of the aorta (e.g., up to and around the aortic arch), to arrive at the aortic valve. Optionally, the insertion site can be dilated prior to insertion of the pump 22 and the introducer sheath. After dilation, the pump 22 and the introducer sheath assembly can be inserted into the vasculature. In other embodiments, a dilator tip can be used. The dilator tip is threaded over the distal end of guide wire. When a dilator is used, a separate pre-dilation step is not required.

FIG. 14 illustrates another embodiment of a system 500 for supporting cardiac function. System 500 is similar to system 10 in that it includes a pump catheter 12 having flexible shaft 14 with a distal end portion configured for advancement into the patient's arterial system (e.g., the femoral or the brachial arteries) across the aortic value 202 and into the left ventricle 204 of the patient's heart 200. Catheter shaft 14 has a proximal end portion coupled to a casing 24 that houses an extracorporeal motor 20 for driving a pump 22 on a distal end portion 16 of catheter 12. In this embodiment, the controller may be housed within the casing 24 and directly coupled to the motor.

System 500 further includes a dilator 502 for creating an internal pathway for a delivery catheter 504 to pass through the patient's arterial system to the aortic valve. Delivery catheter 504 may include a distal tip 510, a substantially rigid coil 508 and a proximal braided section 506 as known in the art. In addition, delivery catheter 504 may further comprise a fluid purge line 512 and a hemostasis valve 514 for ensuring that the internal lumen of delivery catheter 30 remains patent.

Referring now to FIGS. 15-19, a non-contact energy source or drive assembly for transmitting power and/or torque to a transmission line or drive cable will now be described. The energy source and drive cable may be used to power a variety of different devices that are advanced into, or implanted within, a patient, such centrifugal or axial pumps, intra-aortic balloon pumps (IABPs), LVADs, RVADs, biVADS, MCS pumps, veno-arterial extracorporeal membrane oxygenation (VA-ECMO) devices, cardioverter defibrillators, pacemakers, and the like. In an exemplary embodiment, the drive assembly described in FIGS. 15-19 may be used to drive an MCS pump, shown as one of the pumps described herein.

As shown in FIG. 15, the drive assembly comprises a housing 600 and a coupler device 602 sized and configured to removably couple to an internal channel 604 within housing 600. Coupler device 602 functions to removably couple the drive cable to housing 600, which contains the drive assembly (discussed below). Housing 600 may include any suitable configuration, but preferably comprises a relatively small, lightweight construction. In some embodiments, housing 600 and/or coupler device 602 are configured as single-use devices that are disposable after one use by the patient. In other embodiments, these components may be configured for multiple uses by a single patient or multiple patients. For example, in certain embodiments, housing 600 includes a timing control that monitors usage of the drive assembly over, for example, a period of time, or a number of patient uses. The timing control is configured to shut down the drive assembly after it has been used for a certain number of times, or uses.

In certain embodiments, the housing 600 may be configured to be handheld by either the user or the caregiver. In other embodiments, housing 600 may comprise a wearable device that can be attached to, or worn by, the patient. In some embodiments, housing 600 also includes an attachment element (not shown) for attaching housing 600 to a patient.

In one exemplary embodiment, housing 600 includes one or more fixation elements for attaching housing 600 to a substantially planar surface adjacent to, or near, the patient. The fixation elements serve to fix the housing's location as the drive assembly rotates the drive cable. In one such embodiment, the fixation elements comprises first and second bumpers 660, 662 extending from a bottom surface of housing 600. Bumpers 660, 662 may comprise a material, such as rubber or the like, that provides sufficient friction between housing 600 and the planar surface to hold housing 600 in position as the drive cable is rotated. Alternatively, bumpers 660, 662 may comprise other fixation mechanisms, such as magnetic, mechanical or the like.

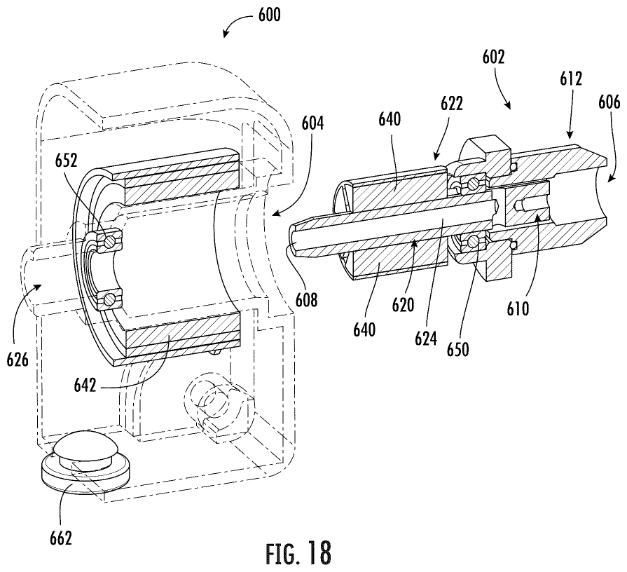

As shown in FIG. 18, coupler device 602 includes a distal opening 606 for receiving a transmission line or drive cable (not shown) and a proximal opening 608 for receiving a fluid line (not shown). Coupler device 602 further includes an internal mating feature 610 for attaching the drive cable to a distal portion 612 of the coupling device. Mating feature 610 is rotatable coupled within distal portion 612 of coupling device 612 such that the drive cable may rotate within device 612.

Mating feature 610 may comprise any suitable coupling device that allows a user to attach the proximal end of a transmission line to device 602. In some embodiments, coupler device 602 is removably coupled to the transmission line. In other embodiments, coupler device 602 may be permanently attached to the transmission line (i.e., the transmission line may be formed integrally with coupler device such that no mating feature 610 is required).

Mating feature 610 is attached to an annular tube 620 within a proximal portion 622 of coupling device 602. Annular tube 620 is also rotatable coupled to proximal portion 622 of the coupling device 602 such that both tube 620 and mating feature 610 may rotate within coupling device 602. Annular tube 620 further includes an internal channel 624 fluidly coupled to proximal opening 606 of coupler device 602 for allowing the passage of fluid through coupling device 602 and into the drive cable. Annular tube 620 preferably extends proximally through a proximal opening 626 within housing 600 for this purpose.

In an exemplary embodiment, the drive cable will include one or more fluid channels that are fluidly coupled to a device that is within the patient, such as an impeller. This allows, for example, purge or flushing fluid to be delivered through housing 600, coupler device 602 and the drive cable and into the impeller. The fluid may be used for a variety of purposes, such as creating positive fluid pressure within housing 600 and coupling device 602 to remove any heat generated by the motor and preventing ingress of blood into the motor and/or to flush the pump, thereby removing heat generated by the impeller and minimizing the formation and/or growth of blood clots within the pump.

Coupler device 602 comprises an external mating feature for attaching device 602 to housing 600. In an exemplary embodiment, the external mating feature comprise an annular projection 632 extending around the exterior surface of device 602 and sized to fit within an annular recess 634 within the distal surface of housing 600 (see FIG. 15). Projection 632 further includes a substantially linear portion 636 that is sized to mate with a substantially linear surface 638 of recess 634. The linear portion 636 of projection 632 is designed to inhibit rotation of coupler device 602 relative to housing 600 when the drive cable is rotated by the magnetic drive assembly (discussed below). Of course, it will be recognized that other linkages may be used to removably attach coupling device 602 to housing 600. For example, coupler device 602 and housing 600 may include annular, cantilever and/or torsional snap-fit linkages or other suitable lock/key coupling mechanisms.

Coupler device 602 comprises one or more magnets 640 extending around annular tube 620. Magnets 640 may comprise permanent magnets that are spaced circumferentially around tube 620. Magnets 640 are coupled to tube 620 such that rotation of magnets 640 causes rotation of tube 620 (and rotation of drive cable therewith). Housing 600 includes a drive mechanism for rotating magnetics 640 and tube 620. In one embodiment, the drive mechanism comprises a magnetic coil 642 within housing 600 that extends around proximal portion 622 of coupler device. Magnetic coil 642 may comprise any suitable wire coil that includes one or more turns designed to produce a magnetic field to rotate magnets 640. In one embodiment, magnetic coil 642 may be driven by an electric current generated by an energy source (not shown). The energy source may be disposed within housing 600, or it may be directly connected to housing 600, or it may be wirelessly connected to housing 600.

In another embodiment, the drive mechanism comprises one or more magnets (not shown) within housing 600. The magnets are rotated around the longitudinal axis of housing 600 by a motor (not shown), and in turn, cause rotation of the coupler device magnets 640. The motor may comprise, for example, any suitable motor designed to function at speeds of up to 35,000 rpms. For example, the motor may include a motor stator (not shown) and a rotatable element, such as a rotor portion of the motor that is configured to be rotated (i.e., driven) by the motor stator. The rotor may be directly coupled to the housing magnets and may be positioned within housing 600, or suitable coupled to housing 600.

Housing 600 and coupling device 602 may further include one or more bearings to resist axial and radial forces applied by the rotating components of coupling device 602 and the drive cable. The bearings may comprise fluid or hydrodynamic bearings, magnetic bearings or mechanical bearings, bushings or the like. In one embodiment, coupling device 602 comprises an annular bearing 650 disposed between proximal and distal portions of coupling device 602. Annular bearing 650 is sized to mate with a suitable recess within housing 600. Housing 600 further comprises an annular bearing 652 disposed proximal of magnets 640 and disposed around tube 620 (see FIG. 15). Bearings 650, 652 are designed to inhibit coupler device 602 from moving axially or radially as the internal components are rotated.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Various alternatives and modifications can be devised by those skilled in the art without departing from the disclosure. Accordingly, the present disclosure is intended to embrace all such alternatives, modifications, and variances. As well, one skilled in the art will appreciate further features and advantages of the present disclosure based on the above-described embodiments. Accordingly, the present disclosure is not to be limited by what has been particularly shown and described, except as indicated by the appended claims.
For example, in a first aspect, a first embodiment is a system for supporting cardiac function. The system comprises a catheter having a shaft with a distal end portion configured for advancing through an aortic valve to a left ventricle of a patient, an impeller coupled to the distal end portion of the catheter, one or more inlets positioned distal of the impeller and coupled to an internal lumen within the catheter, wherein the impeller is configured to draw blood from the left ventricle through the inlets and into the internal lumen, one or more outlets positioned proximal of the impeller and coupled to the internal lumen, wherein the impeller is configured to pump the blood through the outlets and into the aorta, an extracorporeal motor coupled to a proximal end portion of the catheter and a drive member extending through the catheter shaft and coupling the impeller with the motor to rotate the impeller.
A second embodiment is the first embodiment, further comprising a controller coupled to the motor.
A third embodiment is any combination of the first two embodiments, wherein the motor is spaced from the drive member.
A 4^{th} embodiment is any combination of the first 3 embodiments, wherein the motor comprises an energy source for generating a magnetic field, the system further comprising one or more magnets coupled to the drive member.
A 5^{th} embodiment is any combination of the first 4 embodiments, further comprising a processor within the controller, wherein the processor is configured to switch the motor from an activated mode, wherein the motor rotates the impeller, to a deactivated mode, wherein the motor is inoperable to drive the impeller.
A 6^{th} embodiment is any combination of the first 5 embodiments, wherein the processor is configured to switch the motor to the deactivated mode after the motor has operated for a specific period of time.
A 7^{th} embodiment is any combination of the first 6 embodiments, wherein the processor is configured to switch the motor to the deactivated mode after the motor has operated for a specific number of procedures.
An 8^{th} embodiment is any combination of the first 7 embodiments, wherein the drive member comprises a transmission line configured to impart rotational force on the impeller.
A 9^{th} embodiment is any combination of the first 8 embodiments, wherein the drive member comprises a torque cable.
A 10^{th} embodiment is any combination of the first 9 embodiments, further comprising a housing comprising the motor.
An 11^{th} embodiment is any combination of the first 10 embodiments, further comprising: a coupling device for removably coupling the drive member to the housing; and a proximal port on the housing coupled to a fluid line extending through the housing and the drive member.
A 12^{th} embodiment is any combination of the first 11 embodiments, wherein the housing is handheld device
A 13^{th} embodiment is any combination of the first 12 embodiments, wherein the housing comprises an attachment element for coupling to a patient.
A 14^{th} embodiment is any combination of the first 13 embodiments, wherein the housing is a wearable device.
A 15^{th} embodiment is any combination of the first 14 embodiments, wherein the distal end portion of the catheter is formed into a curve.
A 16^{th} embodiment is any combination of the first 15 embodiments, wherein the curve substantially corresponds to a portion of an aortic arch of the patient.
A 17^{th} embodiment is any combination of the first 16 embodiments, wherein the distal end portion of the catheter comprises an atraumatic distal tip.
An 18^{th} embodiment is any combination of the first 17 embodiments, wherein the distal tip is configured to move between a first substantially linear configuration to a second curved configuration.
A 19^{th} embodiment is any combination of the first 18 embodiments, further comprising a flush line having a proximal end configured for coupling to a source of fluid, and a distal opening at the distal end portion of the catheter.
A 20^{th} embodiment is any combination of the first 19 embodiments, wherein the flush line extends through the catheter and the distal opening is proximal of the impeller for delivering fluid through the impeller.
A 21^{st} embodiment is any combination of the first 20 embodiments, further comprising a fluid purge line having a proximal end configured for coupling to a source of fluid, and a distal opening within the motor for generating positive fluid pressure within the motor.
A 22^{nd} embodiment is any combination of the first 21 embodiments, further comprising a fluid bearing on the catheter proximal of the impeller, wherein the fluid bearing is configured to at least partially resist radial forces applied to the impeller by the blood.
A 23^{rd} embodiment is any combination of the first 22 embodiments, wherein the fluid bearing is configured to at least partially resist axial forces applied to the impeller by the blood.
A 24^{th} embodiment is any combination of the first 23 embodiments, further comprising a casing on the distal end portion of the catheter, wherein the impeller is housed within the casing.
A 25^{th} embodiment is any combination of the first 24 embodiments, wherein the fluid bearing comprises a substantially cylindrical end portion of the catheter disposed within the housing proximal to the impeller, wherein the end portion and an inner surface of the housing define an annular gap between.
In another aspect, a first embodiment is a system for supporting cardiac function, the system comprising: a guide catheter having a shaft with a distal end portion configured for advancing through a blood vessel to an aortic arch within the patient; a pump catheter coupled to the distal end portion of the guide catheter, wherein the pump catheter comprises: an impeller; and one or more inlets positioned distal of the impeller and coupled to an internal lumen within the catheter, wherein the impeller is configured to draw blood from the left ventricle through the inlets and into the internal lumen; and one or more outlets positioned proximal of the impeller and coupled to the internal lumen, wherein the impeller is configured to draw the blood through the outlets and into the aorta.
A second embodiment is the first embodiment, wherein the pump catheter comprises a hub and an elongate member extending distally from the hub, wherein the elongate member has a diameter smaller than the hub.
A third embodiment is any combination of the first two embodiments, wherein the hub comprises a tapered distal surface extending radially inward toward the elongate member.
A 4^{th} embodiment is any combination of the first 3 embodiments, wherein the pump catheter is removably coupled to the guide catheter.
A 5^{th} embodiment is any combination of the first 4 embodiments, wherein the proximal end portion of the pump catheter comprises an annular tube sized to slide a distal end portion of the guide catheter.
A 6^{th} embodiment is any combination of the first 5 embodiments, wherein the proximal end portion of the pump catheter extends around the guide catheter proximally of the hub.
A 7^{th} embodiment is any combination of the first 6 embodiments, further comprising a fluid line within the guide catheter having a proximal end configured for coupling to a source of fluid and a distal end fluidly coupled to the pump catheter.
In another aspect, a first embodiment is a system for supporting cardiac function, the system comprising: a catheter having a shaft with a distal end portion configured for advancing through an aortic valve in a patient to a left ventricle; an impeller coupled to the distal end portion of the catheter; and a positioning device on the catheter proximal of the distal end portion and configured to secure the impeller at a target location within the left ventricle.
A second embodiment is the first embodiment, wherein the positioning device comprises a sheath having an outer diameter sized to secure the sheath in position within an artery proximal to an aortic valve.
A third embodiment is any combination of the first 2 embodiments, wherein the sheath is biased into a curved configuration, wherein the curved configuration substantially matches a curve of an aortic arch of the patient.
A 4^{th} embodiment is any combination of the first 3 embodiments, wherein the impeller is coupled to the sheath.
A 5^{th} embodiment is any combination of the first 4 embodiments, wherein the impeller is configured to advance through at least a portion of the sheath to a position distal of the sheath.
A 6^{th} embodiment is any combination of the first 5 embodiments, wherein the sheath has an inner diameter and the impeller is housed within a casing having an outer diameter, wherein the inner and outer diameters are sized to inhibit movement of the impeller within the left ventricle.
A 7^{th} embodiment is any combination of the first 6 embodiments, wherein the positioning device comprises a wire configured to advance through an internal lumen of the catheter and through an opening in the distal end portion of the catheter.
An 8^{th} embodiment is any combination of the first 7 embodiments, wherein the wire has a distal end portion configured to expand into an enlarged configuration upon advancement through the opening of the catheter.
A 9^{th} embodiment is any combination of the first 8 embodiments, wherein the opening of the catheter is distal of the aortic valve and the enlarged configuration of the distal end portion is sized to inhibit proximal movement of the coil through the aortic valve.
A 10^{th} embodiment is any combination of the first 9 embodiments, wherein the impeller is coupled to the wire such that the wire secures the impeller at the target location in the left ventricle.
In another aspect, a first embodiment is a system comprising: a medical device configured for advancement into a target location within a patient; a drive member having a distal end coupled to the medical device and configured to drive the device, the drive member comprising one or more magnets at a proximal end; and an energy source spaced from the drive member and configured to generate a magnetic field to rotate the magnets and the drive member.
A second embodiment is the first embodiment, wherein the energy source is disposed within a housing, and further comprising a coupling device for removably coupling the proximal end of the drive member to the housing.
A third embodiment is any combination of the first 2 embodiments, further comprising a magnetic coil within the housing.
A 4^{th} embodiment is any combination of the first 3 embodiments, wherein a proximal end portion of the drive member extends through an interior of the housing, wherein the coil at least partially surrounds the drive member.
A 5^{th} embodiment is any combination of the first 4 embodiments, further comprising one or more magnets within the housing.
A 6^{th} embodiment is any combination of the first 5 embodiments, wherein the drive member is rotatably coupled to the coupling device, wherein the coupling device comprises a mating feature for non-rotatably coupling the coupling device to the housing.
A 7^{th} embodiment is any combination of the first 6 embodiments, wherein the housing comprises a proximal port configured for attachment to a fluid delivery line and an internal fluid channel.
An 8^{th} embodiment is any combination of the first 7 embodiments, wherein the coupling device comprises an internal passage fluidly coupled to the internal fluid channel of the housing.
A 9^{th} embodiment is any combination of the first 8 embodiments, wherein the drive member comprises a fluid channel coupled to the passage within the coupling device for delivering fluid through the drive member to the device.
A 10^{th} embodiment is any combination of the first 9 embodiments, wherein the device comprises an impeller, wherein the drive member rotates the impeller.
An 11^{th} embodiment is any combination of the first 10 embodiments, wherein the drive member comprises a transmission line
A 12^{th} embodiment is any combination of the first 11 embodiments, wherein the drive member comprises a torque cable.
A 13^{th} embodiment is any combination of the first 12 embodiments, further comprising a catheter configured for percutaneous advancement into a left ventricle of a patient, wherein the device is coupled to a distal end portion of the catheter.
A 14^{th} embodiment is any combination of the first 13 embodiments, wherein the catheter further comprises: one or more inlets positioned distal of the impeller and coupled to an internal lumen within the catheter, wherein the impeller is configured to draw blood from the left ventricle through the inlets and into the internal lumen; and one or more outlets positioned proximal of the impeller and coupled to the internal lumen, wherein the impeller is configured to pump the blood through the outlets and into the aorta.

What is claimed is:

1. A system for supporting cardiac function, the system comprising:
a catheter having a shaft with a distal end portion configured for advancing through an aortic valve to a left ventricle of a patient;
an impeller coupled to the distal end portion of the catheter;
one or more inlets positioned distal of the impeller and coupled to an internal lumen within the catheter, wherein the impeller is configured to draw blood from the left ventricle through the inlets and into the internal lumen.
one or more outlets positioned proximal of the impeller and coupled to the internal lumen, wherein the impeller is configured to pump the blood through the outlets and into the aorta;
an extracorporeal motor coupled to a proximal end portion of the catheter; and
a drive member extending through the catheter shaft and coupling the impeller with the motor to rotate the impeller.

2. The system of claim 1, further comprising a controller coupled to the motor.

3. The system of claim 1, wherein the motor is spaced from the drive member.

4. The system of claim 3, wherein the motor comprises an energy source for generating a magnetic field, the system further comprising one or more magnets coupled to the drive member.

5. The system of claim 2, further comprising a processor within the controller, wherein the processor is configured to switch the motor from an activated mode, wherein the motor rotates the impeller, to a deactivated mode, wherein the motor is inoperable to drive the impeller.

6. The system of claim 5, wherein the processor is configured to switch the motor to the deactivated mode after the motor has operated for a specific period of time.

7. The system of claim 5, wherein the processor is configured to switch the motor to the deactivated mode after the motor has operated for a specific number of procedures.

8. The system of claim 1, further comprising:
a housing comprising the motor;
a coupling device for removably coupling the drive member to the housing; and
a proximal port on the housing coupled to a fluid line extending through the housing and the drive member.

9. The system of claim 1, wherein the distal end portion of the catheter comprises an atraumatic distal tip configured to move between a first substantially linear configuration to a second curved configuration.

10. The system of claim 1, further comprising a flush line having a proximal end configured for coupling to a source of fluid, and a distal opening at the distal end portion of the catheter.

11. The system of claim 10, wherein the flush line extends through the catheter and the distal opening is proximal of the impeller for delivering fluid through the impeller.

12. The system of claim 1, further comprising a fluid purge line having a proximal end configured for coupling to a source of fluid, and a distal opening within the motor for generating positive fluid pressure within the motor.

13. The system of claim 1, further comprising a fluid bearing on the catheter proximal of the impeller, wherein the fluid bearing is configured to at least partially resist radial forces applied to the impeller by the blood.

14. The system of claim 13, wherein the fluid bearing is configured to at least partially resist axial forces applied to the impeller by the blood.

15. A system for supporting cardiac function, the system comprising:
a catheter having a shaft with a distal end portion configured for advancing through an aortic valve in a patient to a left ventricle;
an impeller coupled to the distal end portion of the catheter; and
a positioning device on the catheter proximal of the distal end portion and configured to secure the impeller at a target location within the left ventricle.

16. The system of claim 15, wherein the positioning device comprises a sheath having an outer diameter sized to secure the sheath in position within an artery proximal to an aortic valve.

17. The system of claim 16, wherein the sheath is biased into a curved configuration, wherein the curved configuration substantially matches a curve of an aortic arch of the patient.

18. The system of claim 15, wherein the impeller is configured to advance through at least a portion of the sheath to a position distal of the sheath.

19. The system of claim 18, wherein the sheath has an inner diameter and the impeller is housed within a casing having an outer diameter, wherein the inner and outer diameters are sized to inhibit movement of the impeller within the left ventricle.

20. The system of claim 15, wherein the positioning device comprises a wire configured to advance through an internal lumen of the catheter and through an opening in the distal end portion of the catheter, wherein the wire has a distal end portion configured to expand into an enlarged configuration upon advancement through the opening of the catheter.
